(19) United States
(12) Patent Application Publication
(10) Pub. No.: US 2020/0292396 A1
(43) Pub. Date: Sep. 17, 2020

(54) TEMPERATURE INDICATOR FOR FIBER CONNECTORS AND METHODS REGARDING SAME

(71) Applicant: Boston Scientific Scimed, Inc., Maple Grove, MN (US)

(72) Inventors: Ashkan ARYAEI, Fremont, CA (US); Cassandra VILLICANA, San Jose, CA (US)

(73) Assignee: Boston Scientific Scimed, Inc., Maple Grove, MN (US)

(21) Appl. No.: 16/781,057

(22) Filed: Feb. 4, 2020

Related U.S. Application Data
(60) Provisional application No. 62/818,198, filed on Mar. 14, 2019.

(51) Int. Cl. G01K 11/14; A61B 18/20; A61B 18/00

(52) U.S. Cl. CPC G01K 11/14; A61B 2018/00809; A61B 2018/00678; A61B 18/20

(57) ABSTRACT

A medical laser fiber for receiving laser light and outputting the laser light to a location determined by a user, the fiber including a connector at a first end, and a laser output at a second end, and an indicator located on the connector, the indicator visually changing when a temperature of the connector is above a temperature threshold.

### CROSS-REFERENCE TO RELATED APPLICATIONS

This patent application claims the benefit of priority under 35 U.S.C. § 119 to U.S. Provisional Patent Application No. 62/818,197, filed Mar. 14, 2019, which is herein incorporated by reference in its entirety.

### TECHNICAL FIELD

The present disclosure relates generally to medical/surgical laser systems, and more particularly, to methods and tools for notifying a user that a portion of the system has an elevated temperature.

### BACKGROUND

Medical laser systems are used for a variety of surgical procedures. These procedures may include dusting and/or fragmentation of stones in the kidney, the bladder, and/or the ureter. Medical laser systems are also used to create incisions and to ablate and/or coagulate soft tissues, such as, but not limited to, the prostate.

Medical laser systems generally include a laser generating module connected to an output fiber using a connector. The laser fibers transfer high energy light beams from the laser generating apparatus to patient treatment sites. The high energy of the light beams can leak out from the fiber into the connector and a temperature of the connector can increase over the duration of the fiber use. In some cases, the temperature of the connector increases enough that the connector can burn a user if contacted by the user. The present disclosure may solve the problem set forth above, and/or other problems in the art. The scope of the disclosure, however, is defined by the attached claims and not the ability to solve a specific problem.

### SUMMARY OF THE DISCLOSURE

In one aspect, a medical laser fiber configured to receive laser light and configured to output the laser light to a location determined by a user comprises: a connector at a first end, and a laser output at a second end, and an indicator located on the connector, the indicator configured to visually change when a temperature of the connector is above a temperature threshold.

According to another aspect, a method for determining a temperature of a medical laser fiber of a medical laser system comprises: delivering a laser light through a connector of the medical laser fiber; and determining, in response to a visual change of an indicator on the connector, a temperature of the connector is greater than an elevated temperature threshold.

### BRIEF DESCRIPTION OF THE DRAWINGS

The accompanying drawings, which are incorporated in and constitute a part of this specification, illustrate various exemplary embodiments and together with the description, serve to explain the principles of the disclosed embodiments.

FIG. 1 is a schematic of a medical laser system according to an exemplary embodiment;

FIG. 2 is a perspective view of an output fiber according to an exemplary embodiment;

FIG. 3 is a perspective view of an output fiber connector according to an exemplary embodiment;

FIG. 4 if a perspective view of an output fiber connector according to another exemplary embodiment; and

FIG. 5 is a flow chart of a method according to an exemplary embodiment.

### DETAILED DESCRIPTION

Both the foregoing general description and the following detailed description are exemplary and explanatory only and are not restrictive of the features, as claimed. As used herein, the terms “comprises,” “comprising,” “having,” “including,” or other variations thereof, are intended to cover a non-exclusive inclusion such that a process, method, article, or apparatus that comprises a list of elements does not include only those elements, but may include other elements not expressly listed or inherent to such a process, method, article, or apparatus. In this disclosure, relative terms, such as, for example, “about,” “substantially,” “generally,” and “approximately” are used to indicate a possible variation of ±10% in a stated value or characteristic.

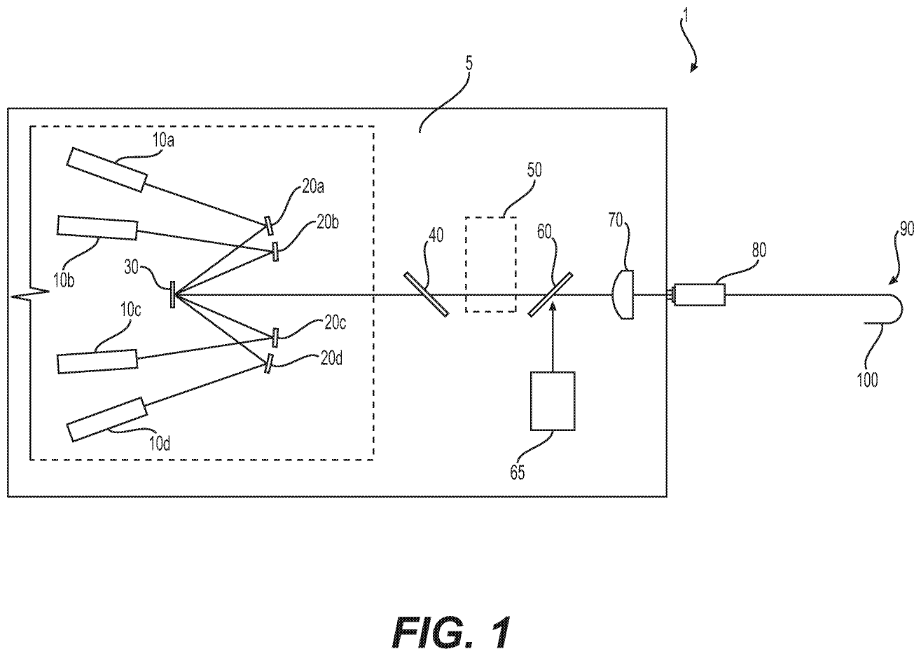

FIG. 1 illustrates an exemplary embodiment of a medical laser system 1. Medical laser system 1 includes a medical laser console 5 having one or more cavities 10 (e.g., cavities 10a-d) (e.g., laser resonators), each cavity capable of outputting an output laser beam (or laser light). The output laser beam from cavities 10a-d is directed to a corresponding relay mirror 20 (e.g., relay mirrors 20a-d). For ease of reference, reference will be made to a single cavity 10a and a single relay mirror 20a. For example, the output laser beam is output from cavity 10a to relay mirror 20a. Each output laser beam is reflected from relay mirror 20a to a Galvo mirror 30. For example, an output laser beam is reflected from relay mirror 20a to Galvo mirror 30. Galvo mirror 30 reflects each output laser beam along a same optical path to a beam splitter 40. A shutter 50 selectively outputs each output laser beam from beam splitter 40 to a beam combiner 60. Beam combiner 60 combines the output laser beams from cavity 10 with an aiming beam from an aiming beam source 65, and passes the combined output laser beam to a coupling lens 70. The aiming beam may be a relatively low power light beam in the visual spectrum that enables an operator to visualize where the output beam from cavity 10 will be fired. Coupling lens 70 couples the output laser beam and matches the output laser beam to an output fiber 90, which is connected to medical laser console 5 by a connector 80, to be transmitted to a delivery location. It is understood that laser console 5 may include any additional or alternative laser generating components known in the art.

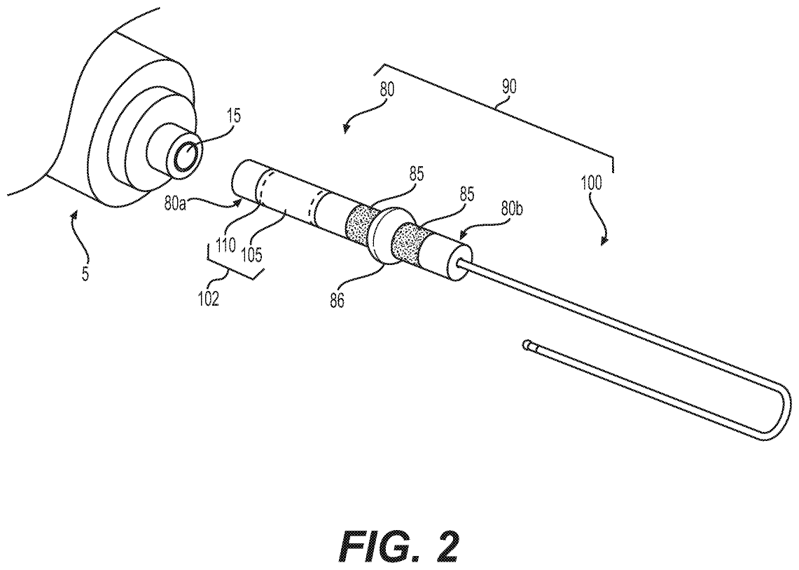

Referring to FIG. 2, connector 80 may be fixed to output fiber 90 in any conventional manner including, but not limited to, laser welding and/or adhesive connections. Connector 80 may also be removably connected to medical laser console 5. For example, connector 80 may include screw threads (not shown) and may be attached to an aperture 15 having corresponding threads on medical laser console 5. Connector 80 is not limited to this connection mechanism and may include any other conventional connection mechanism, e.g., by a snap fit connection to a corresponding aperture on medical laser console 5. Additionally or alternatively, connector 80 could be fixed to medical laser console 5 by, e.g., adhesive, welding, or any attachment now known or later developed.

According to an embodiment, a user may apply a laser energy to one or more locations on or in a patient's body with the aid of output fiber 90. Generally, the user may grasp output fiber 90 and maneuver output fiber 90 inside or outside the body to place a distal tip of output fiber 90 at a location for applying laser light energy. Manipulating output fiber 90 may include contacting connector 80. For example, a user may accidentally contact connector 80 when reaching for and/or when maneuvering output fiber 90. Alternatively or additionally, the user may manipulate connector 80 to attach and/or detach output fiber 90 from medical laser console 5.

With continued reference to FIG. 2, output fiber 90 may include of a glass fiber 100 which may transmit light from medical laser console 5 to the desired output location from a distal end of the output fiber 90 and connector 80 which may connect output fiber 90 to medical laser console 5. According to the disclosure, connector 80 may be formed of a metal or a metal alloy, such as aluminum or an aluminum alloy. It will be understood, however, that connector 80 is not limited to metal and may be any now known or later developed substance suitable for connecting output fiber 90 to medical laser console 5 including, but not limited to, a resin or plastic rated for high temperature use or a ceramic. Similarly, glass fiber 100 is not limited to glass, and may be any conventional material for transmitting laser energy from medical laser console 5 to an output location.

In some embodiments, output fiber 90 may leak light energy to connector 80 because, e.g., output fiber 90 may not be completely efficient. Light energy that leaks into connector 80 may heat connector 80 and cause connector 80 to become hot (a temperature above ambient temperature). For example, prior to using laser medical system 1, connector 80 may be at an ambient, or room, temperature. When laser medical system 1 is activated and laser energy passes from cavity 10 through medical laser console 5 to output fiber 90, light energy may escape from output fiber 90 into connector 80. As light energy escapes output fiber 90 into connector 80, the temperature of connector 80 may increase above the ambient temperature. For example, the temperature of connector 80 may increase from an ambient temperature to a temperature that can injure a user, including burning, causing a user pain, and/or other bodily injury.

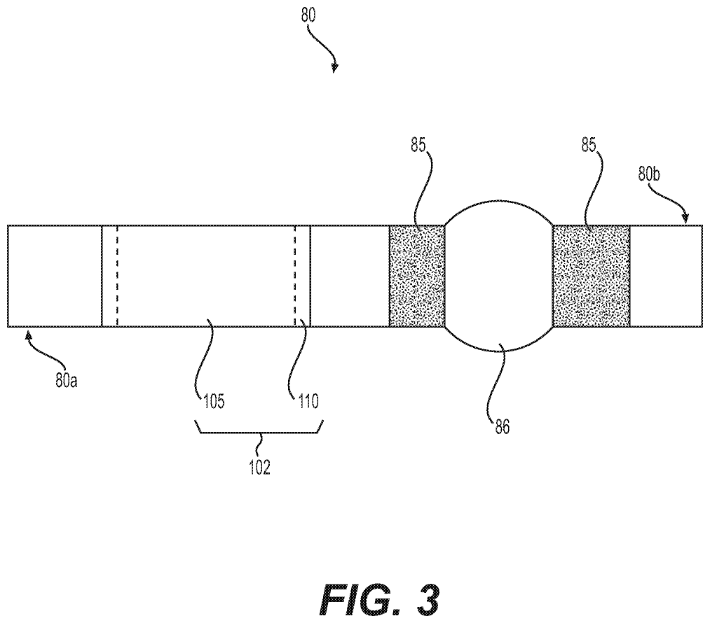

Referring to FIGS. 2 and 3, an example of connector 80 according to the disclosure is illustrated. Connector 80 has a generally cylindrical shape with a smooth outer surface at a proximal end 80a, adjacent the medical laser console 5, and a distal end 80b having two textured gripping portions 85. A ring-shaped member 86 having an outer diameter greater than an outer diameter of proximal end 80a may be provided between gripping portions 85. Ring-shaped member 86 and gripping portions 85 may aid a user in manipulating connector 80, such as providing the user with an area to grip when connecting connector 80 to medical laser console 5. It will be understood that the shape and outer surface of connector 80 are not limited to this configuration. For example, in some embodiments, proximal end 80a may have protrusions and a ring-shaped member while distal end 80b may have a smooth outer surface. Additionally or alternatively, the entire outer surface of connector 80 may be smooth; the entire outer surface of connector 80 may include textured gripping portions; and/or the outer surface of connector 80 may include one or more ridges to facilitate gripping of connector 80. It will be understood by one of skill in the art that the shape and outer surface of connector 80 may be any shape and/or surface finish sufficient to allow a user to manipulate connector 80 and to connect connector 80 to medical laser console 5.

With renewed reference to FIG. 3, connector 80 may include an indicator 102 provided around proximal end 80a. According to an example, indicator 102 is a visual indicator for a user which indicates when connector 80 has reached a certain temperature. For example, indicator 102 may include a first inner radial layer (e.g., colored tape) 105 provided around a periphery of connector 80 and a second outer radial layer (e.g., chromatic layer) 110 provided on top of at least part of first layer 105. First layer 105 may be disposed partially or completely beneath second layer 110. According to the disclosure, second layer 110 may be opaque when second layer 110 is below a temperature threshold, such that a user is unable to view first layer 105. As a temperature of second layer 110 increases to and above a temperature threshold, second layer 110 may become semi-transparent and/or completely transparent. That is, second layer 110 may be formed of a chromatic material that changes from opaque to translucent as a temperature of the chromatic material increases. The chromatic material can be used as an ink or pigment mixed with resin which can either be stamped, brushed, or sprayed as second outer radial layer 110. As discussed herein, the activation temperature of this chromatic material can range from, e.g., approximately 40° C. to approximately 60° C. to avoid user injury. A wide range of materials, including organic and inorganic materials (e.g., inorganic polymers), may be used for the chromatic material. As second layer 110 becomes transparent, a user may view first layer 105 through second layer 110, which may indicate that connector 80 is above a temperature threshold and should not be touched. For example, first layer 105 may be a colored tape, e.g., a red tape, and may signify that a user should not touch connector 80 for safety reasons. It will be understood that first layer 105 may be a paint, ink, or other marking mechanism applied directly to a surface of connector 80.

Connector 80 may be formed of a material that is generally thermally conductive, such that connector 80 is approximately a same temperature across its surface. As connector 80 heats up from an ambient temperature to a temperature above a temperature threshold, second layer 110 heats up at approximately the same rate and has approximately a same temperature as connector 80. According to an embodiment, a temperature threshold may be approximately 40° C. to approximately 70° C., more particularly 60° C. According to an embodiment, the temperature threshold may be equal to or greater than 40°. The temperature threshold may be a temperature at which a user feels discomfort and/or causes injury to a user, such as burning a user's skin when the user contacts connector 80. When connector 80 and second layer 110 reach the temperature threshold, second layer 110 may gradually transition from opaque to semi-transparent and/or completely transparent, thereby informing a user when connector 80 is above the temperature threshold.

Indicator 102 is not limited to a colored tape. For example, first layer 105 may include a word, a phrase, a picture, an emoji, and/or any other graphic to indicate connector 80 is above a temperature threshold. For example, first layer 105 may display the word HOT and, when the temperature of connector 80 and second layer 110 are above a temperature threshold, second layer 110 may become semi-transparent and/or transparent and a user may view the word HOT through the second layer 110. In this manner, a user may become readily aware that connector 80 is too hot to handle and can avoid connector 80.

Additionally or alternatively, second layer 110 may gradually transition from being semi-transparent or transparent to opaque as the temperature of second layer 110 decreases below the temperature threshold. For example, second layer 110 may become semi-transparent of transparent when connector 80 is heated to a temperature above the temperature threshold during a patient treatment using medical laser system 1. After treating a patient using medical laser system 1, medical laser system 1 may be shut down and/or laser energy may stop flowing to fiber 90 through connector 80. Connector 80 may begin to cool to an ambient temperature, which may be below the temperature threshold. As connector 80 cools below the temperature threshold, second layer 110 may transition from semi-transparent or transparent to opaque and may block a user's view of first layer 105. Thus, a user may understand that when first layer 105 is no longer visible, it may be safe to contact connector 80. This may allow a user to disconnect fiber 90 from medical laser console 5 without fear of being injured by a hot connector 80.

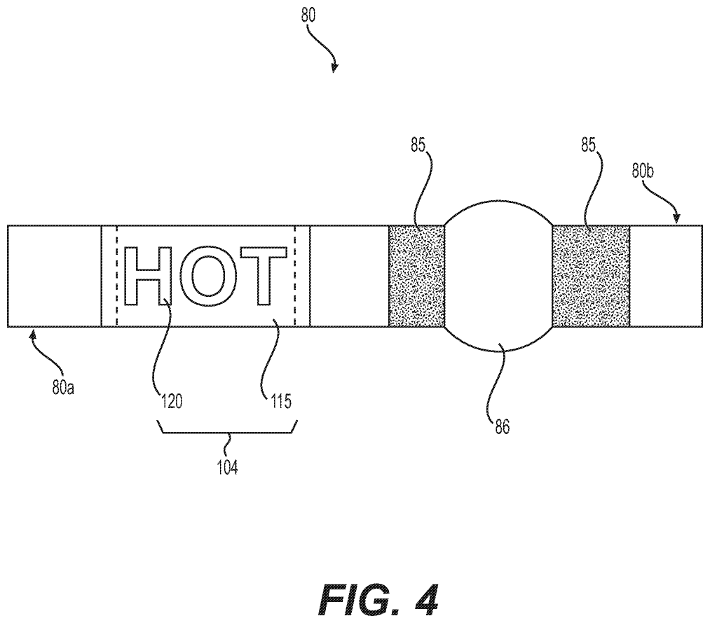

Reference is now made to FIG. 4, which depicts another aspect of the disclosure. Indicator 104 according to an embodiment may include a first portion 115 which is chromatic and a second portion 120 which is non-chromatic. If indicator 104 is below a temperature threshold, indicator 104 may be a first color, pattern, design, etc. In response to connector 80 and, thus, indicator 104 being heated above a temperature threshold, first portion 115 may change color, thereby changing a color, pattern, design, etc. of indicator 104. For example, indicator 104 may be a white color below the temperature threshold. As laser energy passes from medical laser console 5 to fiber 90 through connector 80, the temperature of connector 80 and indicator 104 may increase above the temperature threshold. As indicator increases above the temperature threshold, first portion 115 may change from a white color to a red color while second portion 120 may remain a white color. First portion 115 and second portion 120 may be arranged on indicator 104 such that second portion 120 displays the word HOT on a background, e.g., red background, in response to first portion 115 changing from a white color to a red color, thereby indicating to a user that connector 80 is above the temperature threshold. It will be understood that both first portion 115 and second portion 120 may both be chromatic, or only an area of each of first portion 115 and second portion 120 may be chromatic, and a color of the chromatic portions may change when a temperature thereof is above a temperature threshold.

After dissipation of laser energy from medical laser console 5 is terminated, connector 80 and first portion 115 may begin to cool, causing first portion 115 to transition from the red color to the white color. Once connector 80 and first portion 115 have cooled below the temperature threshold, the entirety of indicator 104 may be a white color and may not display the word HOT. Accordingly, a user may understand that it is safe to contact connector 80. It will be understood that first portion 115 and second portion 120 may be disposed on indicator 104 in any manner to form any word, phrase, symbol, etc. that may indicate to a user that connector 80 is too hot to touch when a temperature of connector 80 is above a temperature threshold.

Additionally or alternatively, indicator 104 may include more than first portion 115 and second portion 120 as indication areas. For example, indicator 104 may include several areas of different colors and/or may have areas having different chromatic parameters. For example, a first area of indicator 104 may change color at a first temperature and a second area of indicator 104 may change temperature at a different, higher temperature. This arrangement may aid in creating different patterns, words, phrases, symbols, etc. to inform a user of gradations in the elevated temperature of the connector 80.

According to yet another embodiment of this disclosure, indicator 102, 104 may be applied using known techniques, e.g., as a paint or an ink, and may be applied directly to connector 80. For example, indicator 102, 104 may be applied directly to connector 80 as a paint and may include multiple layers of chromatic and non-chromatic materials. When a temperature of connector 80 is increased above a temperature threshold, a user may determine connector 80 is too hot to touch based on a change of at least a portion of indicator 102, 104, e.g., a change in color or pattern. Applying indicator 102, 104 to an entirety of connector 80 provides additional visual cues for a user and may allow a user to identify connector 80 is above a temperature threshold at an earlier time. Alternatively or additionally, a chromatic dye may be used in the formation of connector 80 and/or connector 80 may be formed of a material having chromatic properties. Thus, when a temperature of connector 80 is increased above a temperature threshold, the entirety of connector 80 may change color to indicate connector 80 is above a temperature threshold.

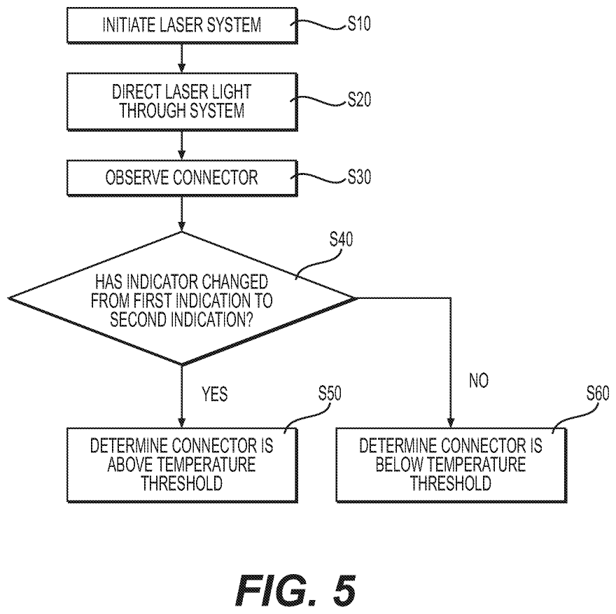

Referring to FIG. 5, a method of determining if connector 80 is greater than a temperature threshold is described. In Step S10, medical laser system 1 may be initiated. This may include powering on medical laser console 5 and initiating any startup functions, including but not limited to safety checks, preparation of laser cavities 10a-d, etc. Once medical laser system 1 is initiated, the user may direct laser light through medical laser system 1 in Step S20. This may include firing a laser light from one or more of laser cavities 10a-d and outputting laser light through fiber 90. As the laser light passes from medical laser console 5 into fiber 90, the laser light passes through connector 80 and may heat connector 80.

In Step S30, a user may observe connector 80, including indicator 102, 104. Indicator 102, 104 may be a first color, including but not limited to a transparent color. As the laser light enters connector 80, connector 80 may be below a temperature threshold and, thus, indicator 102, 104 may be a color that indicates the same.

In Step S40, it is determined whether indicator 102, 104 has changed from a first color to a second color. It will be understood that a changing of color of indicator 102, 104 may include changing from a first color, e.g., blue, to a second color, e.g., red, or may include changing from a first color to a second color, e.g., transparent. Additionally or alternatively, a portion of indicator 102, 104 may change color and/or become transparent. As described herein, indicator 102 may have first layer 105 that is visible to a user when second layer 110 changes from a color to a transparent color. According to another example, indicator 104 may include first portion 115 and second portion 120. As connector 80 increases in temperature above a temperature threshold, first portion 115 may change color.

If a color of indicator 102, 104 changes in Step S50, it may be determined that the temperature of connector 80 is above a threshold and may be too hot for a user to touch. Accordingly, indicator 102, 104 may display an indication, such as the word HOT, to indicate that the user should not touch connector 80. Further, if a color of indicator 102, 104 does not change in Step S60 (or changes back to a non-elevated temperature indication), a user may determine that connector 80 is below a temperature threshold and that connector 80 may be cool enough to touch or handle.

It will be apparent to those skilled in the art that various modifications and variations can be made to the disclosed device without departing from the scope of the disclosure. Other embodiments of the disclosure will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A medical laser fiber configured to receive laser light and configured to output the laser light to a location determined by a user, the fiber comprising:
a connector at a first end, and a laser output at a second end, and
an indicator located on the connector, the indicator configured to visually change when a temperature of the connector is above a temperature threshold.

2. The laser system according to claim 1, wherein the indicator includes a first layer applied to an outer surface of the connector and a second layer provided on at least a portion of the first layer.

3. The laser system according to claim 2, wherein the first layer is completely disposed beneath the second layer.

4. The laser system according to claim 2, wherein the first layer is a non-chromatic material.

5. The laser system according to claim 2, wherein the second layer is a chromatic material.

6. The laser system according to claim 5, wherein the second layer changes from a first color to a second color when the temperature is above the temperature threshold.

7. The laser system according to claim 5, wherein the second color is a transparent color.

8. The laser system according to claim 1, wherein the indicator includes a first portion and a second portion disposed on a surface of the connector.

9. The laser system according to claim 8, wherein at least one of the first portion and the second portion is configured to change from a first color to a second color when the temperature of the connector is above the temperature threshold.

10. The laser system according to claim 8, wherein the first portion is configured to change from the first color to the second color and the second portion is configured to change from a third color to a fourth color, different from the second color, when the temperature of the connector is above the temperature threshold.

11. The laser system according to claim 1, wherein, in response to the temperature being greater than the temperature threshold, the indicator displays one or more of a word, a phrase, a pattern, and a color to indicate the temperature is above the temperature threshold.

12. The laser system according to claim 1, wherein the temperature threshold is equal to or greater than 40° C.

13. The laser system according to claim 1, wherein the connector is formed of a chromatic material.

14. The laser system according to claim 13, wherein a color of the connector is configured to change in response to the temperature being greater than the temperature threshold.

15. The laser system according to claim 2, wherein the first layer and the second layer is one or more of a tape, a paint, a decal, a ceramic, a metal, and a fabric.

16. A method for determining a temperature of a medical laser fiber of a medical laser system, the method comprising:
delivering a laser light through a connector of the medical laser fiber; and
determining, in response to a visual change of an indicator on the connector, a temperature of the connector is greater than an elevated temperature threshold.

17. The method according to claim 16, wherein the indicator includes a first layer applied to an outer surface of the connector and a second layer provided on at least a portion of the first layer, the second layer changing from a first indicia to a second indicia in response to the temperature of the connector being greater than the elevated temperature threshold.

18. The method according to claim 17, wherein the second layer changes from the second indicia to the first indicia in response to the temperature being equal to or less than the elevated temperature threshold.

19. A connector for a medical laser fiber, the connector comprising:
an indicator provided on a surface of the connector;
wherein the indicator includes a first portion and a second portion disposed on a surface of the connector and visible to a user, and at least one of the first portion and the second portion is configured to change from a first color to a second color when the temperature of the connector is above the temperature threshold.

20. The connector according to claim 19, wherein, in response to the temperature being greater than the temperature threshold, the indicator displays one or more of a word, a phrase, a pattern, and a color to indicate the temperature is above the temperature threshold.
